(19) United States
(12) Patent Application Publication
(10) Pub. No.: US 2006/0050145 A1
(43) Pub. Date: Mar. 9, 2006

(54) Imaging device for an endoscope

(75) Inventor: Takashi Tanimoto, Motosu-gun (JP)

Correspondence Address: OLIFF & BERRIDGE, PLC, P.O. BOX 19928, ALEXANDRIA, VA, 22320, US

(73) Assignee: SANYO ELECTRIC CO., LTD., Moriguchi-shi (JP)

(21) Appl. No.: 11/203,247

(22) Filed: Aug. 15, 2005

(30) Foreign Application Priority Data
Sep. 9, 2004 (JP) 2004-262251

(51) Int. Cl. A61B 1/04; H04N 7/18; A62B 1/04; H04N 9/47; A61B 1/06

(52) U.S. Cl. 348/68; 348/65

(57) ABSTRACT

Further miniaturization of capsule-type endoscopes is intended. A CCD image sensor composed of an imaging section, a horizontal transfer section, and an output section is incorporated in a capsule-type endoscope. The inside of a human body is basically in a dark state. Therefore, by setting a desired exposure time on the basis of a light emission period of an LED, the CCD image sensor does not require, separately from light-receiving pixels, vertical shift registers that are shielded from light like the storage sections of frame-transfer-type CCD image sensors and the vertical shift registers of interline-transfer-type CCD image sensors. Since no shielded-from-light regions for maintaining signal charges are necessary, the CCD image sensor can be miniaturized accordingly, which enables further miniaturization of capsule-type endoscopes.

### FIELD OF THE INVENTION

The present invention relates to an imaging device used in an endoscope.

### BACKGROUND OF THE INVENTION

Endoscopes are used for observing objects that are out of reach of human eyesight. Endoscopes are used not only for medical purposes such as observation of internal surfaces of human digestive organs but also for industrial purposes such as observation of the inside of pipes, machines, and structural bodies. Especially, the endoscopes for industrial purposes may be called industrial endoscopes. In general endoscopes, a tube-shaped insertion portion is inserted toward an observation object. The insertion portion is configured in such a manner that an image obtained by a tip portion is guided to the operator side with optical fibers, or that an image sensor is incorporated in a tip portion and an image signal produced by the image sensor is transmitted to the operator side. Since an observation object is basically located in a dark place, the tip portion is provided with a light source for illuminating an observation object.

In recent years, capsule-type endoscopes have been developed and come to be used for observation of human digestive organs etc. The capsule-type endoscopes are such that an image sensor, a light source, their driving circuits, a battery, etc. are incorporated in a small capsule. A subject swallows a capsule-type endoscope, and the capsule-type endoscope transmits, by radio, a picked-up image to the outside of the subject's body while moving through his or her digestive system. Not requiring an insertion portion, such capsule-type endoscopes do not cause a subject pain as would otherwise be caused by insertion of an insertion portion.

To enable entrance into smaller spaces and to reduce the load of a subject (in medical use), further miniaturization of endoscope tips and capsule-type endoscopes is desired.

Endoscopes using a CMOS (complementary metal oxide semiconductor) image sensor or a CCD (charge-coupled device) image sensor have been known so far. CCD image sensors are classified into a frame transfer type, an interline transfer type, and a frame/interline transfer type depending on the method for reading out signal charges obtained at light-receiving pixels. A CCD image sensor of one of these kinds is used in each conventional endoscope.

In CCD image sensors, signal charges accumulated at the individual light-receiving pixels in exposure periods are sequentially moved toward an output section along transfer channels in the device. In conventional CCD image sensors, to suppress mixing of smear components into charge packets during a transfer, transfer channels are provided that are shielded from light and serve to store signal charges after an exposure period. For example, in frame-transfer-type CCD image sensors, a storage section disposed between an imaging section and a horizontal transfer section correspond to such channel regions shielded from light. In interline-transfer-type CCD image sensors, vertical shift registers correspond to such channel regions.

A problem arises that the presence of the channel regions shielded from light restricts the miniaturization of CCD image sensors and hence the miniaturization of the tip portions of endoscopes.

In frame-transfer-type CCD image sensors, a frame transfer from the imaging section to the storage section is performed simultaneous with the completion of an exposure period. This frame transfer is performed at high speed and consumes much power accordingly. In capsule-type endoscopes, for example, this results in a problem that the miniaturization of the battery is restricted and the miniaturization of capsule-type endoscopes themselves is also restricted.

A related technique is disclosed in JP-A-2002-345743.

### SUMMARY OF THE INVENTION

The present invention has been made to solve the above problems, and an object of the invention is therefore to miniaturize a CCD image sensor used in an endoscope and to thereby provide an imaging device for an endoscope capable of realizing an endoscope in which the size of the tip portion of an insertion portion or the capsule size is further reduced.

In an imaging apparatus for an endoscope according to the invention, an imaging device comprises an imaging section in which plural vertical shift registers are arranged in a row direction, individual bits of the vertical shift registers serve as light-receiving pixels for generating signal charges corresponding to incident light, and the signal charges of the respective light-receiving pixels are accumulated and transferred vertically by the vertical shift registers; a horizontal transfer section for receiving, from the imaging section, row by row, the signal charges that are transferred vertically by the vertical shift registers, and for horizontally transferring the received signal charges; and an output section for generating an image signal on the basis of the signal charges that are output from the horizontal transfer section. A driving circuit keeps a light source on in a period corresponding to an exposure period and reads out the image signal by driving the imaging device in a turn-off period of the light source.

### BRIEF DESCRIPTION OF THE DRAWINGS

FIG. 1 is a schematic diagram showing a general configuration of a capsule-type endoscope according to an embodiment of the present invention;

FIG. 2 is a schematic plan view showing a general configuration of a CCD image sensor according to the embodiment of the invention;

FIG. 3 is a flowchart showing an imaging operation in the embodiment of the invention; and

FIG. 4 is a schematic timing chart showing a method for driving an LED and the CCD image sensor with a driving circuit in the imaging operation of FIG. 3 in the embodiment of the invention.

### DESCRIPTION OF THE PREFERRED EMBODIMENTS

An embodiment of the present invention will be hereinafter described with reference to the drawings.

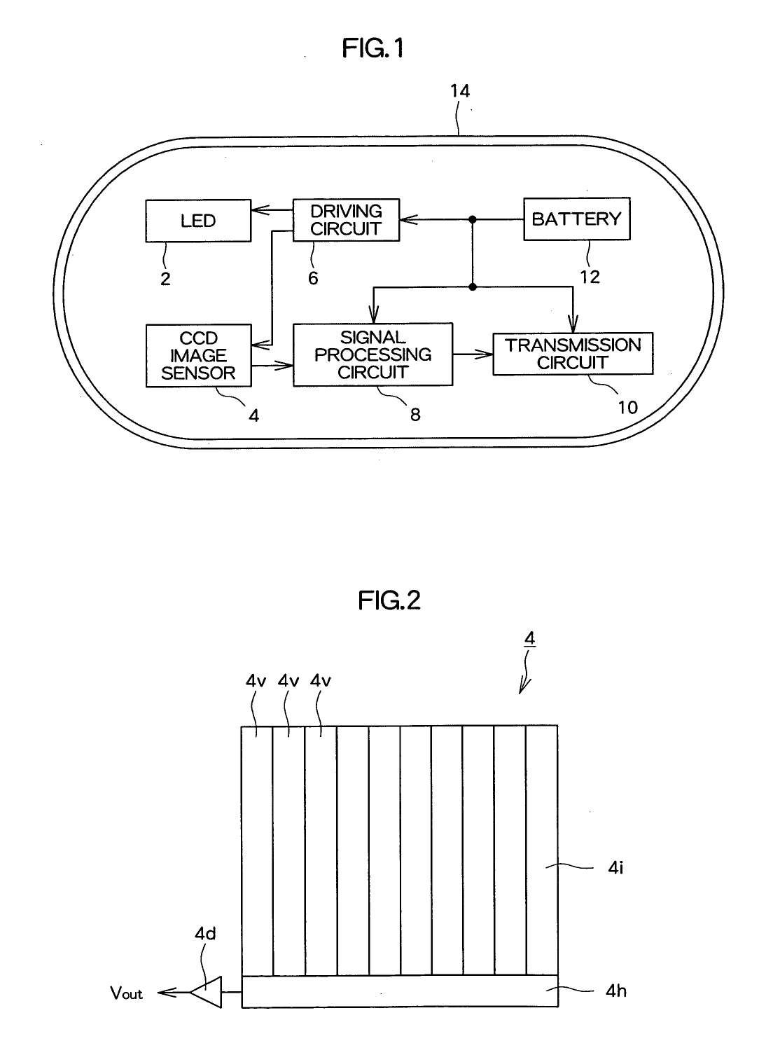

This embodiment is directed to a capsule-type endoscope. FIG. 1 is a schematic diagram showing a general configuration of the capsule-type endoscope according to the embodiment. This capsule-type endoscope, which is to observe, for example, internal surfaces of the digestive system of a subject, is configured in such a manner that an LED (light-emitting diode) 2, a CCD image sensor 4, a driving circuit 6, a signal processing circuit 8, a transmission circuit 10, and a battery 12 are incorporated in a capsule-shaped case 14.

The LED 2 is a light source for emitting light in accordance with a voltage signal supplied from the driving circuit 6. The light emitted from the LED 2 is applied, through a transparent window of the case 14, to an object that is located outside the case 14. Reflection light coming from the illuminated object enters the case 14 through its window.

The CCD image sensor 4 is an imaging device for generating an image signal that reflects an object, and operates according to various clocks supplied from the driving circuit 6. An optical system (not shown) such as a lens is disposed in front of the light-receiving surface of the CCD image sensor 4. The optical system forms an optical image on the light-receiving surface on the basis of reflection light coming from an object, and the CCD image sensor 4 converts an optical image to an image signal Vₒᵤₜ and outputs it.

The driving circuit 6 is supplied with power from the battery 12 and generates various signals (mentioned above) for driving the LED 2 and the CCD image sensor 4.

The signal processing circuit 8 receives an analog image signal Vₒᵤₜ from the CCD image sensor 4, and performs image processing such as correlated double sampling (CDS), automatic gain control (AGC), analog-to-digital conversion (ADC), digital image processing etc.

The transmission circuit 10, which is a circuit for transmitting an image signal by radio, generates a radio signal modulated according to an output of the signal processing circuit 8 and sends it out from an antenna. The battery 12 supplies power to the driving circuit 6 and other circuits.

For example, the case 14 is made of a material that will not be corroded by gastric juices etc. and has a cylindrical, water-proof structure. Assuming a cylindrical shape, the case 14 can easily move in its axial direction with one end portion of its cylindrical shape as the head. Therefore, for example, the LED 2 and the CCD image sensor 4 are disposed so as to be able to access the outside from one end portion and to thereby obtain an image of an object ahead in a traveling direction. The shape of the case 14 shown in FIG. 1 is a schematic cross section including the center axis of the cylindrical shape, and the right end left end portions in FIG. 1 correspond to the end portions of the cylindrical shape. As shown in FIG. 1, the end portions of the cylindrical shape are rounded so that the case 14 can travel smoothly in its axial direction through a human body.

FIG. 2 is a schematic plan view showing a general configuration of the CCD image sensor 4. The CCD image sensor 4 is provided with an imaging section 4i, a horizontal transfer section 4h, and an output section 4d that are formed on the surface of a semiconductor substrate.

The imaging section 4i is composed of plural vertical CCD shift registers (vertical shift registers 4v) that are arranged in the row direction (horizontal direction). Each vertical shift register 4v is provided with plural gate electrodes formed on the semiconductor substrate so as to extend in the row direction. The gate electrodes control potentials of a transfer channel formed in the semiconductor substrate. For example, the driving circuit 6 supplies 3-phase clocks φᵢ to the imaging section 4i. The gate electrodes are 3-phase-driven by the clocks φᵢ, whereby one potential well is formed for each set of three gate electrodes. Signal charges are accumulated in those potential wells and transferred vertically along the transfer channel.

For example, the gate electrodes are made of a material capable of transmitting visible light, such as polysilicon. Each vertical shift register 4v is configured so that light can enter into the semiconductor substrate which corresponds to the transfer channel. Each bit of the vertical shift register 4v functions as a light-receiving pixel for generating a signal charge corresponding to an incident light quantity, and plural light-receiving pixels are arranged in matrix form in the imaging section 4i.

The horizontal transfer section 4h is a CCD shift register, and its individual bits are connected to the outputs of the plural vertical shift registers 4v of the imaging section 4i, respectively. Signal charges of one row are transferred each time from the vertical shift registers 4v to the horizontal transfer section 4h. The horizontal transfer section 4h sequentially transfers the signal charges of one row to the output section 4d.

The output section 4d is composed of a capacitor and an amplifier for reading out its voltage variation. The output section 4d receives, via the capacitor, bit by bit, signal charges that are output from the horizontal transfer section 4h, converts the signal charges to voltage values, and outputs the voltage values in the form of a time-series image signal.

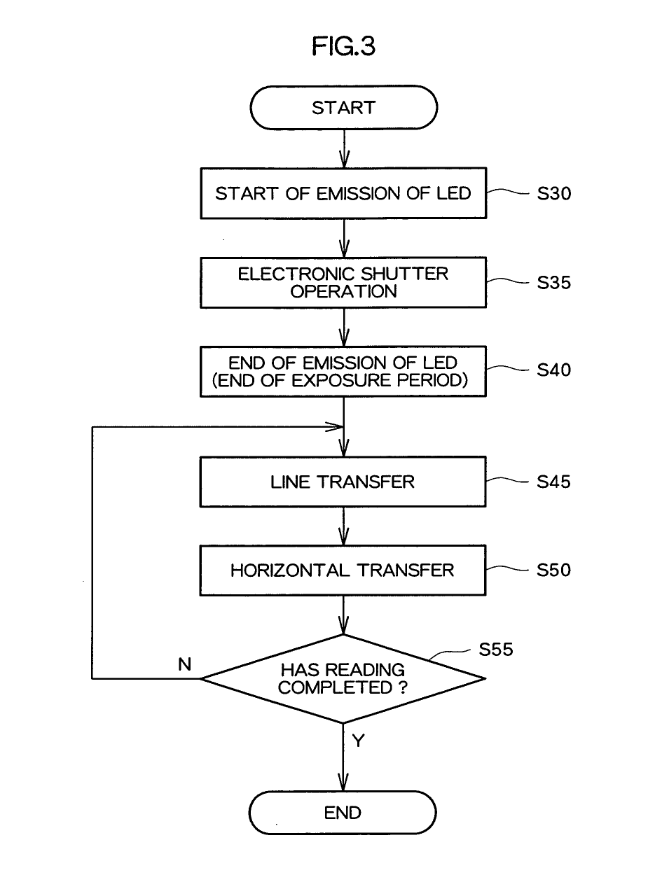

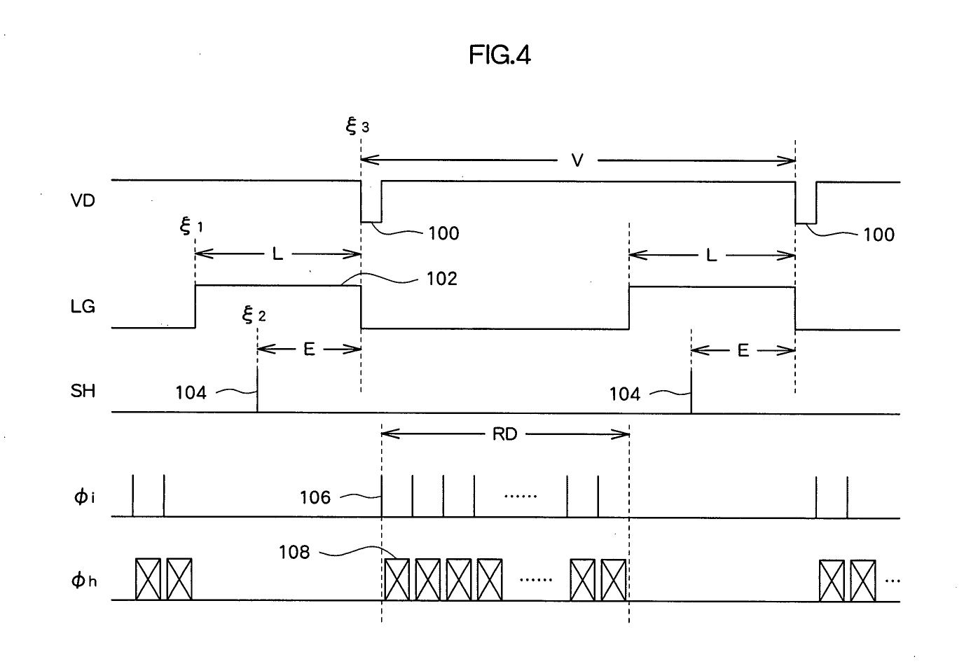

FIG. 3 is a flowchart showing an imaging operation of the device. FIG. 4 is a schematic timing chart showing a method for driving the LED 2 and the CCD image sensor 4 with the driving circuit 6 in the imaging operation of FIG. 3. FIG. 4 shows a vertical sync signal VD, a voltage signal LG that is supplied to the LED 2, clock operation timing of transfer clock signals φᵢ for driving the vertical shift registers 4v of the imaging section 4i, a trigger signal SH for an electronic shutter operation, and clock operation timing of a transfer clock signal φₕ for driving the horizontal transfer section 4h. In FIG. 4, time elapses rightward on the horizontal axis.

In each vertical scanning period V, an operation of reading out signal charges from all the pixels of the imaging section 4i (period RD) and a light-emitting operation of the LED 2 (period L) are performed sequentially. An imaging operation of each frame is started from start of emission S30 (time ξ₁) of the LED 2. For example, the emission start timing 41 of the LED 2 is set to a time that precedes a fall (time ξ₃) of a VD pulse 100 by a prescribed time L. Given a voltage pulse 102 from this time, the LED 2 starts to emit light.

The start of an exposure period E is determined by an electronic shutter operation S35 that is performed in the light emission period L of the LED 2. In the electronic shutter operation, all transfer clocks φᵢ₁-φᵢ₃ are turned off in response to an electronic shutter trigger pulse 104 (time ξ₂), whereby the potential wells of all the pixels are forced to disappear for a prescribed time. As a result, signal charges accumulated in the respective potential wells are ejected from the transfer channels to the back surface of the substrate.

Upon completion of the electronic shutter operation, one, having a prescribed phase, of the clock signals φᵢ(e.g., the clock signal φᵢ₂) is turned on. As a result, potential wells are formed under gate electrodes corresponding to the clock signal φᵢ₂ and accumulation of signal charges is started again. An exposure period E starts from this timing.

The time ξ₂ of generation of the trigger pulse 104 is set so as to precede the fall of the VD pulse 100 by a time E. Since basically no light source exists in a human body except the LED 2, the exposure operation is finished upon turning-off of the LED 2 (step S40). Therefore, in the above control, the end timing of the exposure period E is set the same as the fall of the VD pulse 100 which is the end timing of the light emission period L of the LED 2. The time ξ₂ of the electronic shutter operation which is the start of the exposure period E is determined with the fall of the VD pulse 100 used as a basic point.

For example, the length of the light emission period L may be fixed among frames and the length of the light emission period E can be determined by feedback control based on an exposure level of the preceding frame.

Upon the completion of the exposure period E, an operation of reading out signal charges accumulated in the imaging section 4i (period RD) is started. In the read operation period RD, a line transfer operation S45 of vertically transferring signal charges of one row accumulated in the imaging section 4i and an operation S50 of horizontally transferring, to the output section 4d, the signal charges of one row that have been transferred to the horizontal transfer section 4h by the line transfer are performed alternately. As for the line transfer operation of the vertical shift registers 4v, one-cycle clock operations 106 using the transfer clock signals φᵢ are performed repeatedly in a cycle of a horizontal scanning period H. A one-row horizontal transfer operation using the transfer clock signal φₕ is performed by clock operations 108 whose number of cycles is equal to the number of bits of the CCD shift register as the horizontal transfer section 4h, and is completed in the 1H period.

Line transfer operations S45 and horizontal transfer operations S50 that correspond to one frame are performed repeatedly until completion of reading of signal charges from the imaging section 4i, that is, a number of times that is equal to the number of bits of each vertical shift register 4v (step S55).

The transfer rate of the line transfer performed in the imaging section 4i of the CCD image sensor 4 is lower than that of the frame transfer of frame-transfer-type CCD image sensors. However, in each read operation period RD, the LED 2 does not emit light and hence a good dark state is kept in a human body. Therefore, basically no signal charges generated by received light are added at other transfer-intermediate pixels and image quality deterioration due to smears etc. can be avoided.

The cycle of vertical sync periods VD (i.e., vertical scanning period V), the period RD during which signal charges from all the pixels of the imaging section 4i are read out, and the light emission period L are set so as to satisfy a relationship V≧RD+L. The above-described exposing operation and read operation are performed in each 1V period, whereby a one-frame image signal is output from the CCD image sensor 4.

In the above configuration, each exposure period E is started by an electronic shutter operation. As described above, basically no light source exists in a human body except the LED 2 and hence signal charges accumulated in the individual pixels of the imaging section 4i after completion of a read operation for the preceding frame are due to noise and are basically of very small amounts. It is therefore possible to set each exposure period E by the light emission period L itself without performing an electronic shutter operation. In this case, the exposure level can be adjusted by variably controlling the length of the light emission period L.

In the above capsule-type endoscope, as described above an imaging operation is performed with the CCD image sensor 4 in cooperation with the LED 2. As a result, no shielded portions for maintaining the amounts of signal charges in each read period RD need to be provided in the CCD image sensor 4, which enables miniaturization of the CCD image sensor 4 and hence reduction of the size of the case 14. Further, since no frame transfer is necessary, the power consumption can be reduced. The battery 12 can thus be miniaturized, which also contributes to reduction of the size of the case 14.

On the other hand, the invention can also be applied to endoscopes whose tube-shaped insertion portion is inserted so as to reach an observation object. Also in this case, the miniaturized CCD image sensor 4 can reduce the diameter of the tip portion.

As described above, in the imaging device used in an endoscope, the individual bits of each vertical shift register serves as a light-receiving pixel and signal charges that are output from the vertical shift registers are transferred to the output section by the horizontal transfer section. That is, this imaging device is basically configured in such a manner that the storage sections are omitted in a frame-transfer-type CCD image sensor. In the plural vertical shift registers, signal charges are vertically transferred toward the horizontal transfer section by one row (line transfer) every time a one-cycle horizontal transfer operation of the horizontal transfer section is completed. Since observation objects of endoscopes basically exist in a dark place, signal charges are generated in the vertical shift registers in response to light reception only during a turn-on period of the light source. That is, performing line transfers in the vertical shift registers only during a turn-off period of the light source prevents noise components such as smears from being generated due to light reception at transfer-intermediate portions of the transfer channels. In this manner, in the imaging device for an endoscope, an exposing operation and a read operation are performed on the imaging device in synchronism with turning-on and turning-off of the light source, respectively. Therefore, the imaging device can be miniaturized because it does not require channel regions that are shielded from light like the storage sections of frame-transfer-type CCD image sensors and the vertical shift registers of interline-transfer-type CCD image sensors. The power consumption can be made lower than in frame-transfer-type CCD image sensors by an amount corresponding to the omission of a frame transfer. The battery can be miniaturized accordingly, which makes it easier to implement even smaller capsule-type endoscopes.

The invention can be applied to not only the endoscopes for medical purposes but also the industrial endoscope.

What is claimed is:

1. An imaging apparatus for an endoscope, comprising:
a light source for applying illumination light to an object;
an imaging device for shooting the object;
a driving circuit for driving the light source and the imaging device, the imaging device comprising:
an imaging section in which plural vertical shift registers are arranged in a row direction, individual bits of the vertical shift registers serve as light-receiving pixels for generating signal charges corresponding to incident light, and the signal charges of the respective light-receiving pixels are accumulated and transferred vertically by the vertical shift registers;
a horizontal transfer section for receiving, from the imaging section, row by row, the signal charges that are transferred vertically by the vertical shift registers, and for horizontally transferring the received signal charges; and
an output section for generating an image signal on the basis of the signal charges that are output from the horizontal transfer section,
wherein the driving circuit keeps the light source on in a period corresponding to an exposure period and reads out the image signal by driving the imaging device in a turn-off period of the light source.

2. The imaging apparatus for an endoscope according to claim 1, wherein the diving circuit performs an electronic shutter operation at a time point when the exposure period is started and thereby ejects signal charges collectively that are accumulated in the imaging section.

3. The imaging apparatus for an endoscope according to claim 1, further comprising a battery for supplying power to the light source and the driving circuit, wherein the imaging apparatus is incorporated in a capsule and is to be put in to a living human body.

4. The imaging apparatus for an endoscope according to claim 1, wherein the horizontal transfer section is a horizontal shift register having bits that are connected to output ends of the vertical shift registers, respectively.

5. The imaging apparatus for an endoscope according to claim 1, wherein the driving circuit causes the vertical shift registers of the imaging section to vertically transfer the signal charges by one bit in each cycle in which the horizontal transfer section transfers signal charges of one row.
